(19) United States
(12) Patent Application Publication
(10) Pub. No.: US 2025/0282775 A1
(43) Pub. Date: Sep. 11, 2025

(54) SOLID FORM OF 3-((1R,3R)-1-(2,6-DIFLUORO-4-((1-(3- FLUOROPROPYL)AZETIDIN-3-YL)AMINO)PHENYL)-3-METHYL-1,3,4,9- TETRAHYDRO-2H-PYRIDO[3,4-B]INDOL-2-YL)-2,2-DIFLUOROPROPAN-1-OL TARTRATE

(71) Applicants: Hoffmann-La Roche Inc., Little Falls, NJ (US); Genentech, Inc., South San Francisco, CA (US)

(72) Inventors: Hao HOU, Fremont, CA (US); Laura JERKE, Riehen (CH); Michael KAMMERER, Moehlin (CH)

(73) Assignees: Hoffmann-La Roche Inc., Little Falls, NJ (US); Genentech, Inc., South San Francisco, CA (US)

(21) Appl. No.: 18/859,397

(22) PCT Filed: Apr. 27, 2023
(86) PCT No.: PCT/EP2023/061060

§ 371 Date: Oct. 23, 2024

(30) Foreign Application Priority Data
Apr. 28, 2022 (EP) 22170407.5

(51) Int. Cl. C07D 471/04; A61K 9/48; A61K 31/437

(52) U.S. Cl. CPC C07D 471/04; A61K 9/4816; A61K 9/4858; A61K 9/4866; A61K 31/437

(57) ABSTRACT

The present invention relates to a crystalline form of giredestrant tartrate having an improved particle size distribution, as well as to processes for its preparation, pharmaceutical compositions comprising it, and its use as a medicament.

### FIELD OF THE INVENTION

The present invention relates to a crystalline form of 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol tartrate, to pharmaceutical compositions comprising the same, as well as processes for its preparation and its use as a medicament for the treatment of cancer.

### BACKGROUND OF THE INVENTION

The estrogen receptor (ER) is a ligand-activated transcriptional regulatory protein that mediates induction of a variety of biological effects through its interaction with endogenous estrogens. Endogenous estrogens include 170 (beta)-estradiol and estrones. ER has been found to have two isoforms, ER-α (alpha) and ER-β (beta). Estrogens and estrogen receptors are implicated in a number of diseases or conditions, such as breast cancer, lung cancer, ovarian cancer, colon cancer, prostate cancer, endometrial cancer, uterine cancer, as well as other diseases or conditions. ER-α targeting agents have particular activity in the setting of metastatic disease and acquired resistance. International Patent Applications WO2016097072 and WO2019245974, the entire contents of which are incorporated herein by reference, disclose a number of ER-α targeting agents, including the compound 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol, with recommended INN giredestrant (WHO Drug Information, Vol. 33, No. 4, 2019, Proposed INN: List 122), which is being investigated in clinical trials for the treatment of breast cancer.

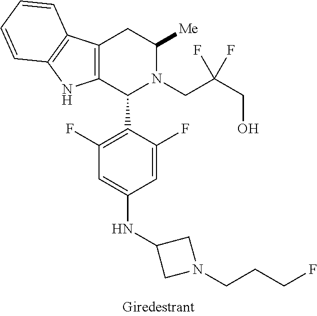 [SMILES: C[C@@H]1Cc2c([nH]c3ccccc23)[C@@H](c2c(F)cc(NC3CN(CCCF)C3)cc2F)N1CC(F)(F)CO]

Among other salt forms, WO2019245974 discloses a tartrate salt of giredestrant having Formula (I), as well as certain crystalline forms thereof.

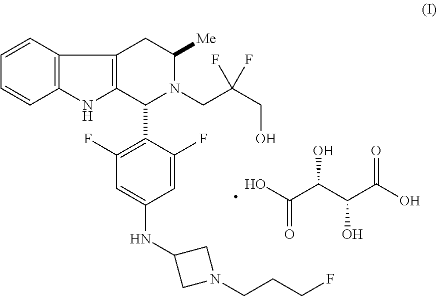

One of the crystalline forms of the tartrate of Formula (I) described WO2019245974 is Form B (hereinafter “Form B”). It has been found that said Form B exhibits a unique mechanical behavior that makes its processing by mechanical compression very difficult. Thus, mechanical compression as applied during tableting of conventional pharmaceutical compositions regularly leads to partial decomposition of the API (see Examples 7 and 8), color changes and lump formation. One way to avoid the detrimental compression forces applied during tableting is to fill the API, or a pharmaceutical composition comprising it, directly into capsules (“direct encapsulation”). However, it has been found that Form B is not well suited for capsule formulations, because it exhibits an inhomogeneous particle size distribution that varies from batch-to-batch—as shown in FIGS. 8 a)-c)—with low bulk density, as well as poor flowability (see Example 9), which hampers processing on an industrial scale.

In summary, Form B is not well suited to provide tablet and capsule formulations on an industrial scale.

Accordingly, there is a high unmet need for a new form of giredestrant that may be formulated into a pharmaceutical product on an industrial scale for administration to patients.

### SUMMARY OF THE INVENTION

The inventors of the present invention have developed a new process for manufacturing crystalline Form B of giredestrant tartrate (see Examples 1 and 2), which reliably and reproducibly affords said Form B with a distinct, monomodal particle size distribution (FIG. 1). Surprisingly, crystalline Form B having said new particle size distribution has improved flow properties and bulk density (see Example 9) compared to crystalline Form B prepared according to the procedures described in WO2019245974, and is very well suited for providing capsule formulations, overcoming the issues outlined above. Capsule formulations comprising the improved drug substance can conveniently be prepared without a granulation step, and without using a binder as an excipient. Furthermore, the capsule formulation of the invention has a lower variability in its dissolution profile than formulations comprising previously known forms of giredestrant, while showing a similar release profile.

In a first aspect, the present invention provides crystalline 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol tartrate of formula (I)

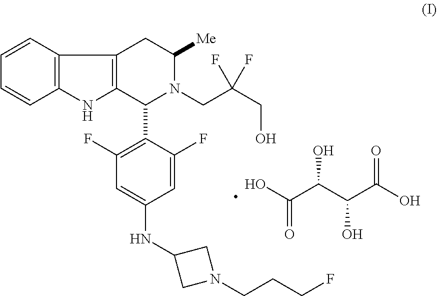

having:
(i) an X-ray powder diffraction pattern comprising peaks at 11.49, 12.54, 19.16, 19.42, or 24.67 [° 2 Theta±0.1° 2 Theta, Cu Kα radiation]; and
(ii) a mono modal particle size distribution with a particle size of D[v,10]=20-54 μm and D[v,90]=38-120 μm.

In further aspects, the present invention provides processes for manufacturing the crystalline form of the invention, formulations comprising the same, and methods of using the same in medical therapy.

### BRIEF DESCRIPTION OF THE FIGURES

FIG. 1 depicts the particle size distribution (PSD) for the crystalline compound of formula (I) according to the present invention.

FIG. 2 depicts a flow chart of the process for manufacturing the pharmaceutical composition according to the invention.

FIG. 3 depicts the API content in sieve fractions of final blends containing the crystalline compound of formula (I) according to the present invention as API (“GPV0137”) or containing the compound of formula (I) in the quality disclosed in WO2019245974 (“GMP0492”) as API.

FIG. 4 depicts plots of impurity with RRT=0.58 observed in API stability samples (uncompressed vs. compressed) stored at 60° C./11% RH for up to 4 weeks.

FIG. 5 depicts plots of impurity with RRT=0.60 (left) and RRT=0.73 (right) observed in stability samples stored at 30° C./65% RH for up to 6 months.

FIG. 6 depicts plots of impurity with RRT=0.60 (left) and RRT=0.73 (right) observed in stability samples stored at 40° C./75% RH for up to 6 months.

FIG. 7 depicts plots of impurity with RRT=0.60 (left) and RRT=0.73 (right) observed in stability samples stored at 60° C./11% RH for up to 6 months.

FIG. 8 a) depicts the particle size distribution of API lot “A” obtained by the process described in WO2019245974 (compare with FIG. 9C of WO2019245974).

FIG. 8 b) depicts the particle size distribution of API lot “B” obtained by recrystallization of API lot “A”.

FIG. 8 c) depicts the particle size distribution of API lot “C” obtained by recrystallization of API lot “A”.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

As used herein, the term “Form B” relates to crystalline Form B of giredestrant tartrate (Formula (I)) as described in WO2019245974, i.a. having an X-ray powder diffraction pattern comprising peaks at 11.49, 12.54, 19.16, 19.42, or 24.67 [° 2 Theta 0.1° 2 Theta, Cu Kα radiation]. In a preferred embodiment, said crystalline Form B has an X-ray powder diffraction pattern comprising the peaks outlined in Table 1:

TABLE 1
Representative XRPD Peaks for Form B of giredestrant tartrate

| 2-Theta (°2θ) | d(Å) |
|---|---|
| 7.684 | 11.4966 |
| 11.491 | 7.6942 |
| 12.54 | 7.053 |
| 14.245 | 6.2123 |
| 15.303 | 5.7851 |
| 15.557 | 5.6912 |
| 16.014 | 5.5301 |
| 16.634 | 5.3252 |
| 17.371 | 5.1009 |
| 18.242 | 4.8593 |
| 19.163 | 4.6278 |
| 19.424 | 4.5662 |
| 19.892 | 4.4597 |
| 20.243 | 4.3833 |
| 21.817 | 4.0705 |
| 22.524 | 3.9442 |
| 22.996 | 3.8644 |
| 23.253 | 3.8223 |
| 23.573 | 3.7711 |
| 24.676 | 3.6049 |
| 25.073 | 3.5487 |
| 25.915 | 3.4353 |

The particle size distribution of crystalline 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol tartrate of the invention was analyzed by laser diffraction as described in the USP <429> for Laser Diffraction Measurement of Particle Size and in the European Pharmacopoeia chapter 2.9.31 Particle size analysis by Laser Diffraction. Details of the apparatus used and measurements are given in Example 3.

As used herein, the terms “hypromellose”, “HPMC” and “hydroxypropyl methylcellulose” refer to cellulose, 2-hydroxypropyl methyl ether (CAS 9004-65-3), and are used interchangeably.

As used herein, the term “HDPE” refers to high density polyethylene.

As used herein, the term “filler” refers to a substance added to a pharmaceutical composition to increase the weight and/or size of the pharmaceutical composition.

Pharmaceutically acceptable fillers are described in Remington's Pharmaceutical Sciences and listed in Handbook of Pharmaceutical Excipients, Sheskey et al., 2017. Non-limiting examples of fillers are starch (e.g., pregelatinized starch), cellulose (e.g., microcrystalline cellulose) and lactose (e.g., lactose monohydrate). Preferred, yet non-limiting examples of fillers are cellulose and lactose.

As used herein, the term “disintegrant” refers to a substance added to a pharmaceutical composition to help break apart (disintegrate), e.g., after administration, and release the active ingredient, such as Form B described herein. Pharmaceutically acceptable disintegrants are described in Remington's Pharmaceutical Sciences and listed in Handbook of Pharmaceutical Excipients, Sheskey et al., 2017. Non-limiting examples of disintegrants are low substituted hydroxypropyl cellulose and croscarmellose sodium. A preferred, yet non-limiting example of a disintegrant is croscarmellose sodium.

As used herein, the term “lubricant” refers to a substance added to a pharmaceutical composition to help reduce the adherence of a granule of powder to equipment surfaces. Pharmaceutically acceptable lubricants are described in Remington's Pharmaceutical Sciences and listed in Handbook of Pharmaceutical Excipients, Sheskey et al., 2017. Non-limiting examples of lubricants are sodium stearyl fumarate and magnesium stearate. A preferred, yet non-limiting example of a lubricant is magnesium stearate.

As used herein, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a cancer.

As used herein, the term “effective amount” refers to an amount of a compound described herein capable of treating a disorder, disease or condition, or symptoms thereof, disclosed herein.

As used herein, the term “patient” refers to animals, such as mammals, including, but not limited to, primates (e.g., humans), cows, sheep, goats, horses, dogs, cats, rabbits, rats, mice, monkeys, chickens, turkeys, quails, or guinea pigs and the like, in one embodiment a mammal, in another embodiment a human. In one embodiment, a subject is a human having or at risk for cancer.

Crystalline Form B of Giredestrant Tartrate with Improved Particle Size Distribution

In a first aspect, the present invention provides crystalline 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol tartrate of formula (I)

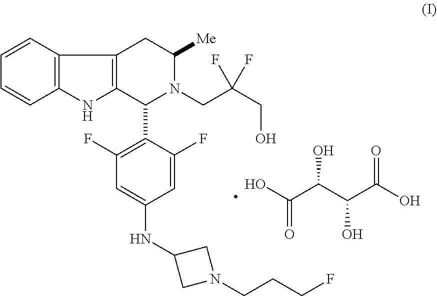

having:
(i) an X-ray powder diffraction pattern comprising peaks at 11.49, 12.54, 19.16, 19.42, or 24.67 [° 2 Theta±0.10 2 [° 2 Theta±0.2° 2 Theta, Cu Kα radiation]; and
(ii) a mono modal particle size distribution with a particle size of D[v,10]=20-54 μm and D[v,90]=38-120 μm.

In one embodiment, said crystalline form has a mono modal particle size distribution with a particle size of D[v,10]=24-50 μm and D[v,90]=45-100 μm.

In one embodiment, said crystalline form has a mono modal particle size distribution with a particle size of D[v,10]=28-46 μm and D[v,90]=50-90 μm.

In a particularly preferred embodiment, said crystalline form has a mono modal particle size distribution with a particle size of D[v,10]=30-42 μm and D[v,90]=56-84 μm.

In a particularly preferred embodiment, said crystalline form has a mono modal particle size distribution with a particle size of D[v,10]=30-42 μm, D[v,50]=40-60 μm and D[v,90]=56-84 μm.

### Process for Manufacturing the New Form

As outlined above, crystalline form B of giredestrant tartrate having a favourable particle size distribution is formed when said crystalline form is prepared by a particular process.

Thus, in a further aspect, the present invention provides a process for manufacturing the crystalline form of giredestrant tartrate described herein, said process comprising the steps of
a) providing a solution of 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol (“free base”) in an organic solvent;
b1) adding the solution of step a) to a solution of tartaric acid in an organic solvent at 15-30° C.; or
b2) adding a solution of tartaric acid in an organic solvent to the solution of step a) at 15-30° C.

In one embodiment, said organic solvent used in the process of the invention is ethanol.

In one embodiment, the process of the invention further comprises:
c) stirring the suspension obtained from b1) or b2) at 15-30° C. for at least 8 hours.

In one embodiment, the temperature in steps b1), b2) and c) of the process of the invention is maintained at 20-25° C.

In a preferred embodiment, the temperature in steps b1), b2) and c) of the process of the invention is maintained at 20° C.

In one embodiment, the concentration of the free base in the solution provided in step a) of the process of the invention is about 13 to 19%-w/w.

In one embodiment, the concentration of tartaric acid in the solution used in step b1) or b2) of the process of the invention is about 8 to 12%-w/w.

In one embodiment of the process of the invention, step b1) comprises:
b1a) adding a first portion of the solution of step a) to a solution of tartaric acid in an organic solvent;
b1b) seeding the solution of step b1a) with the crystalline compound of formula (I) according to claim 1; and
b1c) adding the remainder of the solution of step a) to the mixture of step b1b); and step b2) comprises:
b2a) adding a first portion of a solution of tartaric acid in an organic solvent to the solution of step a);
b2b) seeding the solution of step b2a) with the crystalline compound of formula (I) according to claim 1; and b2c) adding a second portion of a solution of tartaric acid in an organic solvent to the mixture of step b2b).

In one embodiment of the process of the invention:
(i) said first portion of the solution of step a) in step b1a) amounts to about 10-30% of the total amount of solution of step a); and
(ii) said first portion of a solution of tartaric acid in an organic solvent in step b2a) amounts to about 20-30% of the total amount of solution of tartaric acid in an organic solvent that is used in step b2).

In one aspect, the present invention provides a crystalline compound of formula (I) as described herein, when obtained by the processes described herein.

### Pharmaceutical Compositions

In one aspect, the present invention provides a pharmaceutical composition for oral administration comprising the crystalline compound of formula (I) as described herein and one or more pharmaceutically acceptable excipients selected from fillers, disintegrants and lubricants.

In one embodiment, the pharmaceutical composition according to the invention comprises the crystalline compound of formula (I) as described herein and one or more fillers, a disintegrant and a lubricant.

In one embodiment, the pharmaceutical composition according to the invention comprises the crystalline compound of formula (I) as described herein, and:
(i) a first filler;
(ii) a second filler;
(iii) a disintegrant; and
(iv) a lubricant.

In a preferred embodiment:
(i) said first filler is microcrystalline cellulose;
(ii) said second filler is lactose monohydrate;
(iii) said disintegrant is croscarmellose sodium; and
(iv) said lubricant is magnesium stearate.

In one embodiment:
(i) the weight of said first filler represents 33±1% of the total weight of the composition;
(ii) the weight of said second filler represents 10±1% of the total weight of the composition;
(iii) the weight of said disintegrant represents 5±1% of the total weight of the composition;
(iv) the weight of said lubricant represents 0.5±1% of the total weight of the composition; and
(v) the weight of said crystalline compound of formula (I) represents 51.5±1% of the total weight of the composition.

In a preferred embodiment, the pharmaceutical composition according to the invention comprises said compound of formula (I) in an amount of 38.62 mg (equivalent to 30 mg of the “free base”).

In one aspect, the present invention provides a capsule for oral administration containing the pharmaceutical compositions described herein.

In one embodiment, said capsule is made of hypromellose.

In a particularly preferred embodiment, the pharmaceutical composition according to the invention is:

| | | Quantity per Unit | |
|---|---|---|---|
| | | Dose (measure of | |
| Component | Function | weight/capsule) | |
| Giredestrant Tartrate | Active | 38.620 mg | |
| | | (equivalent to 30 | |
| | | mg of the free base) | |
| Microcrystalline Cellulose | Filler | 24.755 | mg |
| Lactose Monohydrate | Filler | 7.500 | mg |
| Croscarmellose Sodium | Disintegrant | 3.750 | mg |
| Magnesium Stearate | Lubricant | 0.375 | mg |
| Target Capsule Fill | | 75.000 | mg |
| Weight | | | |
| HPMC Capsule size 3 | Capsule shell | 47.000 | mg |
| Total Capsule Weight | | 122.000 | mg |

### Manufacturing Process of Pharmaceutical Compositions

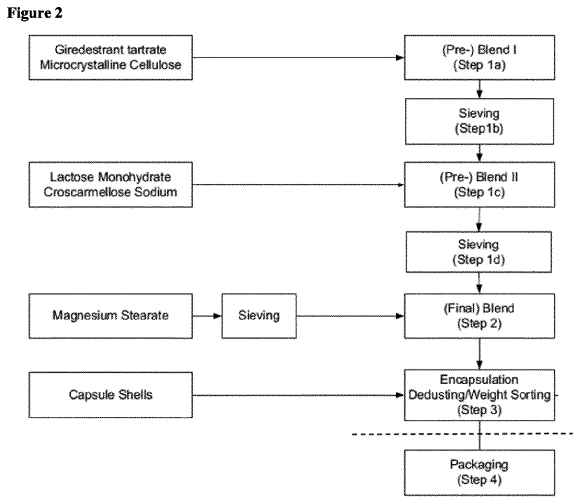

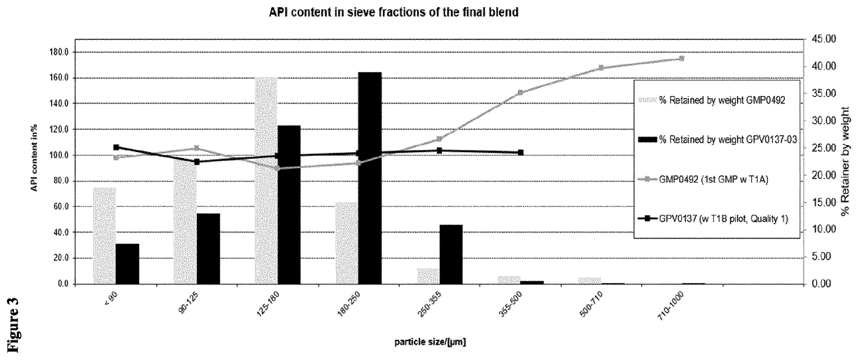

Present invention further provides a process for the manufacture of pharmaceutical compositions as described herein. In particular the present invention provides a process for the manufacture of pharmaceutical compositions according to FIG. 2.

In one aspect, the present invention provides a process for making the pharmaceutical compositions described herein, comprising:
a) combining and blending the crystalline compound of formula (I) described herein and a first filler;
b) sieving the blend obtained in step a);
c) adding a second filler and a disintegrant to the blend obtained in step b);
d) sieving the blend obtained in step c);
e) sieving a lubricant;
f) adding the sieved lubricant from step e) to the blend obtained in step d); and
g) blending the mixture obtained in step f).

In one embodiment, said sieving in steps b) and d) is performed using a conical mill.

In one embodiment, said sieving in step e) is performed using a sieve with a mesh size of 0.5-1.0 mm.

In one embodiment, the process for making the pharmaceutical compositions of the invention further comprises:
(h) transferring the final blend obtained in step g) into capsules.

### Uses

A particular aspect of the present invention relates to crystalline Form B as defined above for use as a medicament.

A further aspect of the invention relates to crystalline Form B as defined herein, as well as to pharmaceutical compositions comprising the same, for use in the treatment of cancer.

In a further aspect, the present invention provides a method for treating cancer in a patient having said cancer, said method comprising administering an effective amount of crystalline Form B described herein, or a pharmaceutical composition described herein to the cancer patient.

In a further aspect, the present invention provides the use of crystalline Form B as defined herein, as well as of pharmaceutical compositions described herein, in a method for treating cancer in a patient having said cancer.

In a further aspect, the present invention provides the use of crystalline Form B as defined herein in the manufacture of a medicament for treating cancer in a patient having said cancer.

In one embodiment, said cancer is selected from lung cancer, ovarian cancer, endometrial cancer, prostate cancer, uterine cancer, or breast cancer.

In a preferred embodiment, said cancer is breast cancer.

In one embodiment, said cancer is breast cancer selected from hormone receptor positive breast cancer, HER2-positive breast cancer, and triple negative breast cancer.

In one embodiment, said cancer is breast cancer selected from HER2-negative, HER2-positive breast cancer, and triple negative breast cancer.

In one embodiment, said cancer is metastatic breast cancer.

In one embodiment, the compound or pharmaceutical composition of the invention is administered as a component of adjuvant therapy.

In one embodiment, the compound or pharmaceutical composition of the invention is administered as a component of neoadjuvant therapy.

In one embodiment, said cancer is breast cancer which is stage 0, I, II, III, or IV.

In one embodiment, the patient has had previous treatment with one or more anti-cancer agents or radiation therapy.

In one embodiment, the patient has had surgery prior to treatment with Form B of the invention.

In one embodiment, Form B of the invention is administered in combination with one or more of radiation therapy, hormone therapy, or an anti-cancer agent.

In one embodiment, Form B of the invention is administered in combination with one or more anti-cancer agents.

In one embodiment, said anti-cancer agents are selected from doxorubicin, pegylated liposomal doxorubicin, epirubicin, paclitaxel, albumin-bound paclitaxel, docetaxel, 5-fluorouracil, cyclophosphamide, cisplatin, carboplatin, vinorelbine, capecitabine, gemcitabine, ixabepilone, eribulin, olaparib, methotrexate, anastrozole, exemestane, toremifene, letrozole, tamoxifen, 4-hydroxy tamoxifen, raloxifene, droloxifene, trioxifene, keoxifene, ftutamide, nilutamide, bicalutamide, lapatinib, vinblastine, goserelin, leuprolide, pegfilgrastim, filgrastim, and venetoclax,

In one embodiment, said anti-cancer agents are selected from an AKT inhibitor, a CDK4/6 inhibitor, a PARP inhibitor, and an aromatase inhibitor.

In one embodiment, said anti-cancer agent is abemaciclib, ribociclib, or palbociclib.

In one embodiment, said anti-cancer agent is abemaciclib.

In one embodiment, said anti-cancer agent is ribociclib.

In one embodiment, said anti-cancer agent is palbociclib.

In one embodiment, said anti-cancer agent is ipatasertib.

In one embodiment, said anti-cancer agent is everolimus or fulvestrant.

In one embodiment, said anti-cancer agent is ado-trastuzumab emtansine, trastuzumab, pertuzumab, or atezolizumab.

In one embodiment, said anti-cancer agent is alemtuzumab, bevacizumab, cetuximab, panitumumab, rituximab, tositumomab, or a combination thereof.

### EXAMPLES

The following examples are provided for illustration of the invention. They should not be considered as limiting the scope of the invention, but merely as being representative thereof.

### Example 1—Manufacture of Crystalline Giredestrant Tartrate

3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol tartrate (9.0 kg crude, as obtained from the process described in WO2019245974, Example 8, [0550], 13.4 mol, 1.0 eq, hereinafter “tartrate”) was suspended in tert-Butylmetylether (TBME) at ambient temperature. 5%-w/w aqueous sodium hydroxide solution (22.4 kg, 28 mol NaOH, 2.1 eq) was added at ambient temperature to afford a solution of 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol (hereinafter “free base”). The aqueous phase was discarded; the organic phase was washed with water to remove salts and filtered via charcoal. Subsequently, a solvent swap from TBME to EtOH was performed by vacuum distillation. The final concentration of the free-base in EtOH at the end of the solvent exchange was adjusted to 18%-w/w. 20% of this solution was added to a solution of tartaric acid (2.1 kg, 14.0 mol, 1.1 eq) in ethanol (17.9 kg) at 20-25° C. The resulting solution was seeded with the tartrate and afterwards the final amount of the ethanolic free-base solution was added at 20-25° C. The resulting suspension was stirred at 20-25° C. for about 10 hours. The suspension was filtered and the filter-cake was washed two times with ethanol (9 kg each) and dried at 50° C. in vacuo (10 mbar) overnight. The tartrate was obtained as yellowish solid (8.5 kg, 94% yield).

### Example 2—Manufacture of Crystalline Giredestrant Tartrate

3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol tartrate (5.5 kg crude, as obtained from the process described in WO2019245974, Example 8, [0550], 8.2 mol, 1.0 eq, hereinafter “tartrate”) was suspended in tert-Butylmetylether (TBME) at ambient temperature. 5%-w/w aqueous sodium hydroxide solution (2.1 eq, 13.8 kg, 17.2 mol NaOH) was added at ambient temperature to afford a solution of 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol (hereinafter “free base”). The aqueous phase was discarded; the organic phase was washed two times with water and filtered via charcoal. Subsequently, a solvent swap from TBME to EtOH was performed by vacuum distillation. The final concentration of the free-base in EtOH at the end of the solvent exchange was adjusted to 16%-w/w.

In parallel, an ethanolic solution of tartaric acid (1.3 kg, 8.7 mol, 1.1 eq) in ethanol (12.0 kg) at ambient temperature was prepraed. 25% of this solution was added to above mentioned free base solution at 20-25° C. The resulting solution is seeded with the tartrate and afterwards the final amount of the ethanolic tartaric acid solution is added at 20-25° C. The resulting suspension is stirred at 20-25° C. for at least 8 hours. The final suspension is then optionally wet-milled to control the number of excessively large agglomerates, if any. The suspension is filtered and the filter-cake is washed two times with ethanol (2 kg each) and dried at 50° C. in vacuo (10 mbar) overnight. The tartrate is obtained as yellowish solid (5.0 kg, 91% yield).

### Example 3—General Procedure for Particle Size Distribution (PSD) Measurement

For the measurement, a Malvern MasterSizer 3000 device from Malvern, UK, coupled with a liquid dispersion unit Hydro MV®, Malvern, UK was used. The crystalline Giredestrant tartrate as obtained from Example 1 or 2 was dispersed in dispersion medium consisting in technical grade n-heptane with 0.2% w/w.-% Span 85 (Sorbitane trioleate, e.g. Fluka/Sigma Cat. Nr. 85549 or equivalent) non-saturated.

Procedure: the crystalline Giredestrant tartrate is directly added in the Hydro MV dispersion unit and stirred at 2500 rpm achieving an appropriate optical concentration (between 5% and 25% optical concentrations).

Measurement: The test dispersion was measured and the cumulative volume dispersion was determined using the laser diffraction instrument mentioned above in accordance with the instruction manual. Measurements were performed after 2 minutes stirring time. The Fraunhofer approximation was used for the particle diameters calculations, Opaque Particle was used for the Particle type and General purpose was used for the Analysis model. The background and the measuring duration were set at 30 seconds.

Three independent samples were measured one time. The average particle size distribution in volume at the undersize values of 10%, 50% and 90% (×10, ×50 and ×90) percentiles were evaluated from the cumulative distribution.

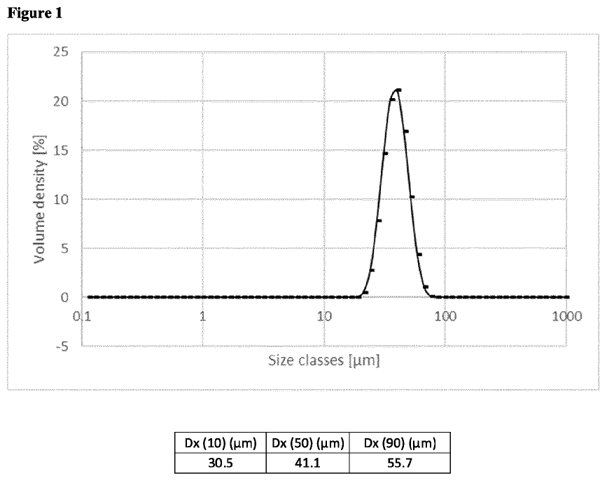

The resulting particle size distribution is shown in FIG. 1.

### Example 4—General Procedure for Preparation of Capsule Formulation

Capsule formulations comprising the crystalline compound of formula (I) according to the invention were prepared as described in the flow diagram of FIG. 2 and following the detailed procedure below.

Step 1a: Giredestrant tartrate and microcrystalline cellulose were combined in one container and blended.

Step 1b: Blend 1a was sieved using a conical mill.

Step 1c: Lactose monohydrate and croscarmellose sodium were added to the blend 1b and blended.

Step 1d: Blend 1c was sieved using a conical mill.

Step 2: Magnesium stearate was sieved through a sieve having a mesh size of 0.5 mm (0.5-1.0 mm), added to the blend from step 1d and blended.

Step 3: The final blend was transferred into empty size 3 HPMC capsules.

Step 4: The capsules were packaged in HDPE bottles with desiccant.

### Example 5—30 mg Capsule Formulation

| | | Quantity per Unit | |
|---|---|---|---|
| | | Dose (measure of | |
| Component | Function | weight/capsule) | |
| Crystalline Compound of | Active | 38.620 mg | |
| Formula (I) | | (equivalent to 30 | |
| | | mg of the free base) | |
| Microcrystalline Cellulose | Filler | 24.755 | mg |
| Lactose Monohydrate | Filler | 7.500 | mg |
| Croscarmellose Sodium | Disintegrant | 3.750 | mg |
| Magnesium Stearate | Lubricant | 0.375 | mg |
| Target Capsule Fill Weight | | 75.000 | mg |
| HPMC Capsule size 3 | Capsule shell | 47.000 | mg |
| Total Capsule Weight | | 122.000 | mg |

All excipients used in the formulation are compendial (Ph. Eur. and/or USP/NF) grade.

### Reference Example 6—Alternative Capsule Formulation

Also provided is a capsule formulation for oral administration consisting of the same ingredients as the capsule formulation of Example 5, with the exception of hypromellose. All excipients used in the formulation are compendial (Ph. Eur. and/or USP/NF) grade. The dose strength of the capsule formulation is preferably 30 mg (free base equivalent).

### Reference Example 6—Immediate Release Tablet Formulation

Also provided is an immediate release tablet formulation for oral administration consisting of giredestrant tartrate, microcrystalline cellulose, lactose, croscarmellose sodium, colloidal silicon dioxide and magnesium stearate. All excipients used in the formulation are compendial (Ph. Eur. and/or USP/NF) grade. The dose strength of the capsule formulation is preferably 50 mg or 10 mg (free base equivalent).

### Example 7—Stability Data for API

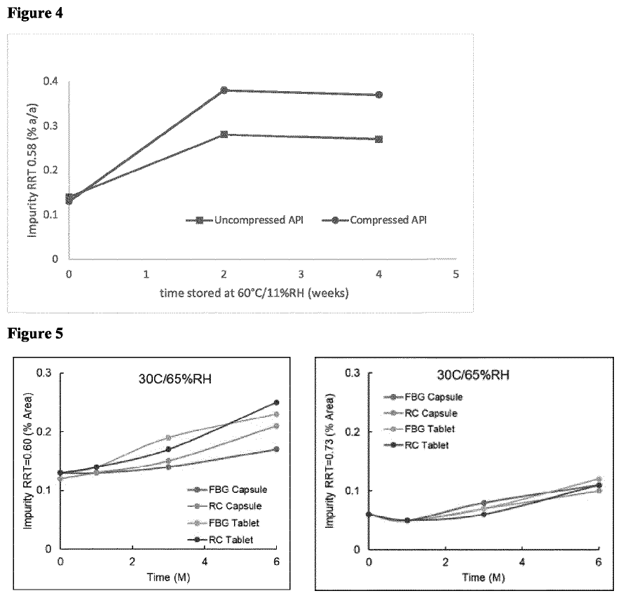

The effect of compression on API degradation was investigated. Samples of uncompressed API (neat API powder as-is) and compressed API (compact) were aged at 60° C./11% RH with containers open for up to 1 month. Samples were assayed by HPLC (see Example 10). FIG. 4 shows the levels of the impurity at RRT=0.58 observed in the samples stored at 60° C./11% RH for up to 4 weeks. The growth of the impurity at RRT=0.58 was observed in both uncompressed and compressed API samples, with faster growth rate observed in compressed samples. The results show that the level of degradation products increased more significantly in compressed samples than uncompressed samples, indicating that the API possesses liabilities to compression force.

### Example 8—Stability Data for 100 mg Capsule and Tablet Formulations

### Abbreviations:

RRT: relative retention time
RH: relative humidity
FBG fluid bed granulation
RC roller compaction
DP drug product

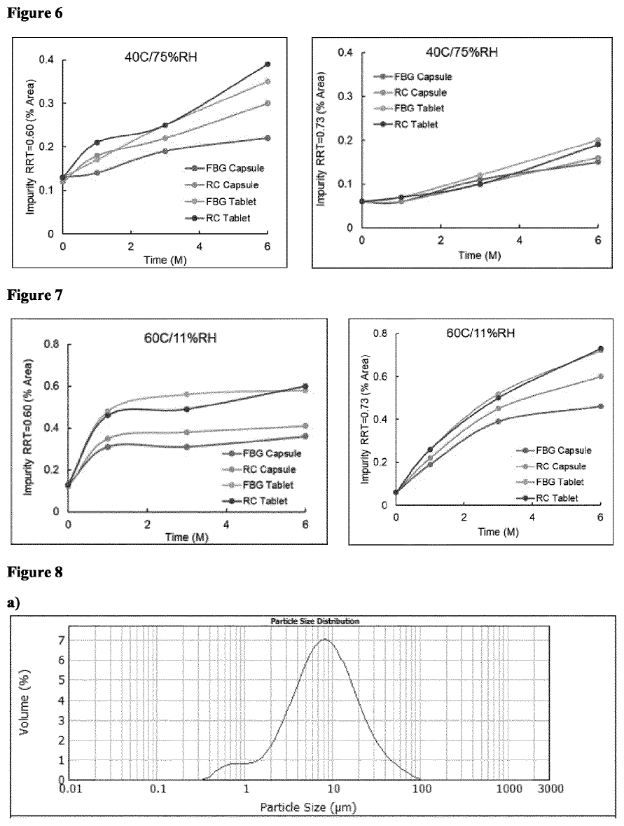

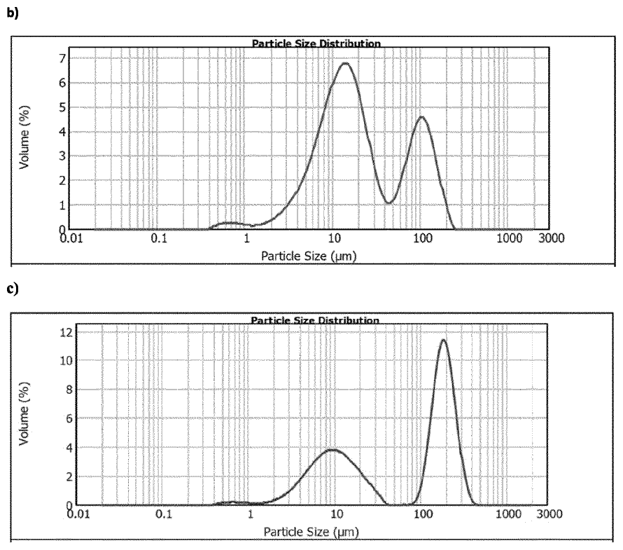

Chemical stability of capsule and tablet formulations was monitored by HPLC (see Example 10) at the initial point, 1, 3, and 6 months. Stability data are listed in the Tables 2-4. The impurity profiles of capsule and tablet formulations at RRT=0.60 and RRT=0.73 observed in stability samples stored at 30° C./65% RH, 40° C./75% RH, and 60° C./11% RH for up to 6 months are shown in FIGS. 5, 6 and 7, respectively. At 30° C./65% RH and 40° C./75% RH, a linear growth of the impurity RRT=0.60 was observed for all samples, but the growth rate was the slowest for FBG capsule. The impurity at RRT=0.73 did not grow as fast as the impurity at RRT=0.60 under the same storage conditions. Its growth rate appeared to be slower for capsules than tablets. At the condition of 60° C./11% RH, the growth of impurity at RRT=0.60 plateaued after 3 months, except for RC tablet. The impurity at RRT=0.73 continued to grow under this condition, whereas starting to plateau for FBG capsule.

Stability results indicate that capsules appeared to be more chemically stable than tablets, with FBG capsule performing the best followed by RC capsule. Comparable growth rates of the impurity at RRT=0.60 was observed from FBG tablet and RC tablet, and it was significantly faster than those observed from capsules. Therefore, the impact of compression force on DP degradation was consistent with previous findings for the API (see Example 7). In summary, the higher the compression force applied during DP manufacturing, the faster the DP degradation would occur.

TABLE 2
1-month Purity Results for 100 mg Capsule and Tablet (FBG and RC)

| | FBG Capsule | | | | | FBG Tablet | | | | |
|---|---|---|---|---|---|---|---|---|---|---|
| RRT | | | 30/ | 40/ | 60/ | | | 30/ | 40/ | 60/ |
| (% a/a) | T0 | 5C | 65 | 75 | 11 | T0 | 5C | 65 | 75 | 11 |
| API | 99.28 | 99.43 | 99.41 | 99.36 | 99.05 | 99.25 | 99.41 | 99.35 | 99.21 | 98.69 |
| Epimer | 0.25 | 0.21 | 0.22 | 0.22 | 0.21 | 0.27 | 0.23 | 0.26 | 0.33 | 0.30 |
| 0.19 | 0.08 | 0.07 | 0.07 | 0.07 | 0.07 | 0.07 | 0.07 | 0.07 | 0.07 | 0.07 |
| 0.58 | 0.13 | 0.13 | 0.13 | 0.14 | 0.31 | 0.13 | 0.12 | 0.14 | 0.17 | 0.48 |
| 0.70 | | 0.05 | 0.06 | 0.05 | 0.08 | | 0.06 | 0.06 | 0.06 | 0.10 |
| 0.73 | 0.06 | 0.05 | 0.05 | 0.06 | 0.19 | 0.06 | 0.05 | 0.05 | 0.07 | 0.26 |
| 1.04 | 0.14 | | | | | 0.15 | | | | |
| 1.06 | 0.07 | 0.07 | 0.07 | 0.09 | 0.10 | 0.07 | 0.07 | 0.07 | 0.10 | 0.10 |

| | RC Capsule | | | | | RC Tablet | | | | |
|---|---|---|---|---|---|---|---|---|---|---|
| RRT | | | 30/ | 40/ | 60/ | | | 30/ | 40/ | 60/ |
| (% a/a) | T0 | 5C | 65 | 75 | 11 | T0 | 5C | 65 | 75 | 11 |
| API | 99.30 | 99.51 | 99.38 | 99.39 | 99.03 | 99.22 | 99.43 | 99.35 | 99.22 | 98.74 |
| Epimer | 0.16 | 0.13 | 0.24 | 0.15 | 0.16 | 0.29 | 0.19 | 0.23 | 0.30 | 0.27 |
| 0.19 | 0.07 | 0.07 | 0.07 | 0.07 | 0.07 | 0.07 | 0.07 | 0.07 | 0.07 | 0.07 |
| 0.58 | 0.12 | 0.13 | 0.13 | 0.18 | 0.35 | 0.13 | 0.12 | 0.14 | 0.21 | 0.46 |
| 0.70 | 0.04 | 0.06 | 0.06 | 0.05 | 0.08 | | 0.05 | 0.06 | 0.06 | 0.10 |
| 0.73 | 0.06 | 0.05 | 0.05 | 0.06 | 0.22 | 0.06 | 0.05 | 0.05 | 0.07 | 0.26 |
| 1.04 | 0.17 | | | | | 0.15 | | | | |
| 1.06 | 0.07 | 0.07 | 0.07 | 0.09 | 0.08 | 0.07 | 0.08 | 0.09 | 0.08 | 0.10 |

TABLE 3
3-month Purity Results for 100 mg Capsule and Tablet (FBG and RC)

| RRT | | | | | | | | | | | | | | | | | | | | |
|---|---|---|---|---|---|---|---|---|---|---|---|---|---|---|---|---|---|---|---|---|
| (% | FBG Capsule | | | | | FBG Tablet | | | | | RC Capsule | | | | | RC Tablet | | | | |
| a/a) | T0 | 5C | 30/65 | 40/75 | 60/11 | T0 | 5C | 30/65 | 40/75 | 60/11 | T0 | 5C | 30/65 | 40/75 | 60/11 | T0 | 5C | 30/65 | 40/75 | 60/11 |
| API | 99.28 | 99.29 | 99.26 | 99.17 | 98.72 | 99.25 | 99.23 | 99.20 | 98.96 | 98.00 | 99.30 | 99.32 | 99.24 | 99.19 | 98.47 | 99.22 | 99.25 | 99.09 | 99.00 | 98.14 |
| Epi- | 0.25 | 0.23 | 0.24 | 0.24 | 0.21 | 0.27 | 0.32 | 0.27 | 0.38 | 0.33 | 0.16 | 0.24 | 0.28 | 0.22 | 0.22 | 0.29 | 0.31 | 0.37 | 0.33 | 0.32 |
| mer | | | | | | | | | | | | | | | | | | | | |
| 0.19 | 0.08 | 0.09 | 0.09 | 0.11 | 0.09 | 0.07 | 0.09 | 0.10 | 0.10 | 0.10 | 0.07 | 0.09 | 0.09 | 0.10 | 0.09 | 0.07 | 0.10 | 0.09 | 0.11 | 0.10 |
| 0.58 | 0.13 | 0.13 | 0.14 | 0.19 | 0.31 | 0.13 | 0.12 | 0.19 | 0.25 | 0.56 | 0.12 | 0.13 | 0.15 | 0.22 | 0.38 | 0.13 | 0.12 | 0.17 | 0.25 | 0.49 |
| 0.59 | | | | | 0.05 | | | | | 0.09 | | | | | 0.07 | | | | | 0.11 |
| 0.66 | | | | | | | | | | 0.10 | | | | | | | | | | |
| 0.70 | | | | | | | | | | 0.08 | 0.04 | | | | 0.09 | | | | | 0.13 |
| 0.73 | 0.06 | 0.07 | 0.08 | 0.11 | 0.39 | 0.06 | 0.06 | 0.07 | 0.12 | 0.52 | 0.06 | 0.06 | 0.07 | 0.10 | 0.45 | 0.06 | 0.05 | 0.06 | 0.10 | 0.50 |
| 1.04 | 0.14 | 0.14 | 0.14 | 0.14 | 0.15 | 0.15 | 0.13 | 0.13 | 0.13 | 0.14 | 0.17 | 0.13 | 0.13 | 0.13 | 0.15 | 0.15 | 0.13 | 0.14 | 0.15 | 0.14 |
| 1.06 | 0.07 | 0.04 | 0.04 | 0.05 | 0.08 | 0.07 | 0.04 | 0.04 | 0.05 | 0.07 | 0.07 | 0.04 | 0.04 | 0.04 | 0.08 | 0.07 | 0.04 | 0.08 | 0.08 | 0.08 |

TABLE 4
6-month Purity Results for 100 mg Capsule and Tablet (FBG and RC)

| RRT | | | | | | | | | | | | | | | | |
|---|---|---|---|---|---|---|---|---|---|---|---|---|---|---|---|---|
| (% | FBG Capsule | | | | FBG Tablet | | | | RC Capsule | | | | RC Tablet | | | |
| a/a) | T0 | 30/65 | 40/75 | 60/11 | T0 | 30/65 | 40/75 | 60/11 | T0 | 30/65 | 40/75 | 60/11 | T0 | 30/65 | 40/75 | 60/11 |
| API | 99.28 | 99.27 | 99.22 | 98.66 | 99.25 | 99.16 | 98.90 | 97.89 | 99.30 | 99.36 | 99.19 | 98.50 | 99.22 | 99.14 | 98.86 | 97.97 |
| Epimer | 0.25 | 0.32 | 0.25 | 0.17 | 0.27 | 0.35 | 0.34 | 0.28 | 0.16 | 0.20 | 0.21 | 0.16 | 0.29 | 0.33 | 0.35 | 0.24 |
| 0.19 | 0.08 | 0.08 | 0.08 | 0.07 | 0.07 | 0.08 | 0.08 | 0.07 | 0.07 | 0.07 | 0.08 | 0.07 | 0.07 | 0.08 | 0.09 | 0.07 |
| 0.54 | | | | | | | | | | | | | | | | 0.05 |
| 0.60 | 0.13 | 0.17 | 0.22 | 0.36 | 0.13 | 0.23 | 0.35 | 0.58 | 0.12 | 0.21 | 0.30 | 0.41 | 0.13 | 0.25 | 0.39 | 0.60 |
| 0.61 | | | | | | | | 0.06 | | | | | | | | 0.05 |
| 0.62 | | | | | | | | 0.05 | | | | | | | | |
| 0.70 | | | | 0.18 | | | 0.06 | 0.23 | 0.04 | | | 0.19 | | | 0.06 | 0.24 |
| 0.73 | 0.06 | 0.11 | 0.15 | 0.46 | 0.06 | 0.12 | 0.20 | 0.72 | 0.06 | 0.10 | 0.16 | 0.60 | 0.06 | 0.11 | 0.19 | 0.73 |
| 1.04 | | | | | 0.07 | 0.06 | 0.07 | | 0.17 | | | | 0.15 | | | |
| 1.06 | 0.07 | 0.05 | 0.07 | 0.10 | | | | 0.12 | 0.07 | 0.06 | 0.06 | 0.08 | 0.07 | 0.05 | 0.07 | 0.08 |

### Example 9—Improved Manufacturability

The following table illustrates the improved manufacturability properties of the crystalline API that is prepared according to the process of the present invention (lot “BS2008SA02”). The E1 lot was prepared according to the process described in WO2019245974.

| | | | | | Bulk Density | Tapped Density |
|---|---|---|---|---|---|---|
| API Lot | D10 (μm) | D50 (μm) | D90 (μm) | BET SA (m2/g) | (g/mL) | (g/mL) |
| 711-005-5467- | 2 | 8 | 27 | 2.9 | 0.31 | 0.41 |
| 001 (E1) | | | | | | |
| BS2008SA02 | 30 | 41 | 56 | 1.7 | 0.57 | 0.64 |

### Example 10—HPLC Method

The following HPLC method was used to measure the RRCs described herein:

| Instrument | Agilent 1260 HPLC system with gradient elution and UV detector | | |
|---|---|---|---|
| Column | Sigma-Aldrich Ascentis Express C18, 2.7 μm, 3 mm × 150 mm | | |
| Flow rate | 0.5 mL/min | | |
| Mobile phase A | 10 mM potassium phosphate buffer, pH 6.5 | | |
| Mobile phase B | ACN:IPA = 70:30 (v/v) | | |
| Run time | 51 minutes | | |
| | Time (min.) | Mobile Phase A, % | Mobile Phase B, % |
| Gradient | 0.0 | 80 | 20 |
| | 5.0 | 62 | 38 |
| | 21.0 | 50 | 50 |
| | 36.0 | 20 | 80 |
| | 41.0 | 20 | 80 |
| | 41.1 | 80 | 20 |
| | 51.0 | 80 | 20 |

| | |
|---|---|
| Injection volume | 5 μL |
| Needle wash | ACN:D.I. water = 50:50, v/v |
| Autosampler | 5° C. |
| Column temp. | 15° C. |
| Detection | UV @ 230 nm (Note: for identification, use DAD @ 230 nm (bandwidth: |
| | 4 nm) and collect the UV spectrum between 200 nm and 400 nm) |

1. Crystalline 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol tartrate of formula (I) 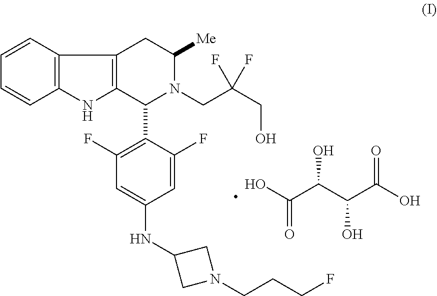
having:
(i) an X-ray powder diffraction pattern comprising peaks at 11.49, 12.54, 19.16, 19.42, or 24.67 [° 2 Theta±0.1° 2 Theta, Cu Kα radiation]; and
(ii) a mono modal particle size distribution with a particle size of D[v,10]=20-54 μm and D[v,90]=38-120 μm.

2. A process for manufacturing the crystalline compound of formula (I) according to claim 1, said process comprising the steps of:
a) providing a solution of 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoro-propan-1-ol (“free base”) in an organic solvent;
b1) adding the solution of step a) to a solution of tartaric acid in an organic solvent at 15-30° C.; or
b2) adding a solution of tartaric acid in an organic solvent to the solution of step a) at 15-30° C.

3. The process for manufacturing according to claim 2, wherein said organic solvent is ethanol.

4. The process for manufacturing according to any one of claims 2 or 3, further comprising:
c) stirring the suspension obtained from b1) or b2) at 15-30° C. for at least 8 hours.

5. The process for manufacturing according to any one of claims 2 to 4, wherein the temperature in steps b1), b2) and c) is maintained at 20-25° C.

6. The process for manufacturing according to claim 5, wherein the temperature in steps b1), b2) and c) is maintained at 20° C.

7. The process for manufacturing according to any one of claims 2 to 6, wherein the concentration of the free base in the solution provided in step a) is about 13 to 19%-w/w.

8. The process for manufacturing according to any one of claims 2 to 7, wherein the concentration of tartaric acid in the solution used in step b1) or b2) is about 8 to 12%-w/w.

9. The process for manufacturing according to any one of claims 2 to 8, wherein:
step b1) comprises:
b1a) adding a first portion of the solution of step a) to a solution of tartaric acid in an organic solvent;
b1b) seeding the solution of step b1a) with the crystalline compound of formula (I) according to claim 1; and
b1c) adding the remainder of the solution of step a) to the mixture of step b1b); and
step b2) comprises:
b2a) adding a first portion of a solution of tartaric acid in an organic solvent to the solution of step a);
b2b) seeding the solution of step b2a) with the crystalline compound of formula (I) according to claim 1; and
b2c) adding a second portion of a solution of tartaric acid in an organic solvent to the mixture of step b2b).

10. The process for manufacturing according to claim 9, wherein:
(i) said first portion of the solution of step a) in step b1a) amounts to about 10-30% of the total amount of solution of step a); and
(ii) said first portion of a solution of tartaric acid in an organic solvent in step b2a) amounts to about 20-30% of the total amount of solution of tartaric acid in an organic solvent that is used in step b2).

11. The crystalline compound of formula (I) according to claim 1, when obtained by the process according to any one of claims 2 to 10.

12. A pharmaceutical composition for oral administration comprising the crystalline compound of formula (I) according to claim 1 or 11 and one or more pharmaceutically acceptable excipients selected from fillers, disintegrants and lubricants.

13. The pharmaceutical composition according to claim 12, comprising the crystalline compound of formula (I) according to claim 1 or 11 and one or more fillers, a disintegrant and a lubricant.

14. The pharmaceutical composition according to claim 12, wherein said pharmaceutically acceptable excipients comprise:
(i) a first filler;
(ii) a second filler;
(iii) a disintegrant; and
(iv) a lubricant.

15. The pharmaceutical composition according to claim 14, wherein:
(i) said first filler is microcrystalline cellulose;
(ii) said second filler is lactose monohydrate;
(iii) said disintegrant is croscarmellose sodium; and
(iv) said lubricant is magnesium stearate.

16. The pharmaceutical composition according to any one of claims 14 or 15, wherein:
(i) the weight of said first filler represents 33±1% of the total weight of the composition;
(ii) the weight of said second filler represents 10±1% of the total weight of the composition;
(iii) the weight of said disintegrant represents 5±1% of the total weight of the composition;
(iv) the weight of said lubricant represents 0.5±1% of the total weight of the composition; and
(v) the weight of said crystalline compound of formula (I) represents 51.5±1% of the total weight of the composition.

17. The pharmaceutical composition according to any one of claims 12 to 16, wherein said compound of formula (I) is present in an amount of 38.62 mg (equivalent to 30 mg of the “free base”).

18. A capsule for oral administration containing the pharmaceutical composition according to any one of claims 12 to 17.

19. The capsule according to claim 18, wherein said capsule is made of hypromellose.

20. The pharmaceutical composition according to any one of claims 12-17, which is:

| | | Quantity per Unit | |
|---|---|---|---|
| | | Dose (measure of | |
| Component | Function | weight/capsule) | |
| Giredestrant Tartrate | Active | 38.620 mg | |
| | | (equivalent to 30 | |
| | | mg of the free base) | |
| Microcrystalline Cellulose | Filler | 24.755 | mg |
| Lactose Monohydrate | Filler | 7.500 | mg |
| Croscarmellose Sodium | Disintegrant | 3.750 | mg |
| Magnesium Stearate | Lubricant | 0.375 | mg |
| Target Capsule Fill | | 75.000 | mg |
| Weight | | | |
| HPMC Capsule size 3 | Capsule shell | 47.000 | mg |
| Total Capsule Weight | | 122.000 | mg |

21. A process for making the pharmaceutical composition according to any one of claims 12 to 20, comprising:
a) combining and blending the crystalline compound of formula (I) according to claim 1 or 11 and a first filler;
b) sieving the blend obtained in step a);
c) adding a second filler and a disintegrant to the blend obtained in step b);
d) sieving the blend obtained in step c);
e) sieving a lubricant;
f) adding the sieved lubricant from step e) to the blend obtained in step d); and
g) blending the mixture obtained in step f).

22. The process according to claim 21, wherein said sieving in steps b) and d) is performed using a conical mill.

23. The process according to any one of claims 21 or 22, wherein said sieving in step e) is performed using a sieve having a mesh size of 0.5-1.0 mm.

24. The process according to any one of claims 21 to 23, further comprising:
(h) transferring the final blend obtained in step g) into capsules.

25. The compound according to claim 1 or 11, for use as a medicament.

26. A method for treating cancer in a patient having said cancer, said method comprising administering an effective amount of the compound according to claim 1 or 11, or the pharmaceutical composition according to any one of claims 12 to 20 to the cancer patient.

27. The compound according to claim 1 or 11, or the pharmaceutical composition according to any one of claims 12 to 20, for use in a method according to claim 26.

28. Use of the compound according to claim 1 or 11, or of the pharmaceutical composition according to any one of claims 12 to 20, in a method according to claim 26.

29. Use of the compound according to claim 1 or 11 in the manufacture of a medicament for treating cancer in a patient having said cancer.

30. The invention as described hereinbefore.
